(19) United States
(12) Patent Application Publication
(10) Pub. No.: US 2023/0251184 A1
(43) Pub. Date: Aug. 10, 2023

(54) SENSOR SYSTEM AND METHOD OF DETECTING TARGET SUBSTANCE

(71) Applicant: TIANMA JAPAN, LTD., Kawasaki (JP)

(72) Inventors: Koki TAKAHASHI, Kawasaki (JP); Nobuya SEKO, Kawasaki (JP); Satoshi MIURA, Kawasaki (JP); Koji SHIGEMURA, Kawasaki (JP); Ken SUMIYOSHI, Kawasaki (JP); Haruki YAMANE, Akita-shi (JP); Satoshi YANASE, Akita-shi (JP); Kiyoshi YAMAKAWA, Akita-shi (JP); Shingo TAKAHASHI, Akita-shi (JP)

(73) Assignee: TIANMA JAPAN, LTD., Kawasaki (JP)

(21) Appl. No.: 18/162,899

(22) Filed: Feb. 1, 2023

(30) Foreign Application Priority Data
Feb. 2, 2022 (JP) 2022-014720
Oct. 13, 2022 (JP) 2022-164929
Jan. 16, 2023 (JP) 2023-004209

(51) Int. Cl. G01N 21/21; G01N 21/77; G01N 33/00

(52) U.S. Cl. CPC G01N 21/21; G01N 21/77; G01N 33/005; G01N 2021/7773; G02B 5/0808

(57) ABSTRACT

A sensor system includes a sensing element, an illumination optical system including a light source, the illumination optical system being configured to obliquely illuminate the sensing element, and a detector device configured to detect light reflected off the sensing element. The sensing element includes a chemical sensing layer configured to change in an optical characteristic in response to contact with a target substance, a reflection layer configured to reflect at least part of incident light, and an intermediate layer located between the reflection layer and the chemical sensing layer. The detector device is configured to separately detect p-polarized light and s-polarized light reflected off the sensing element.

### CROSS-REFERENCE TO RELATED APPLICATIONS

This non-provisional application claims priority under 35 U.S.C. §119(a) on Patent Application No. 2022-014720 filed in Japan on Feb. 2, 2022, Patent Application No. 2022-164929 filed in Japan on Oct. 13, 2022 and Patent Application No. 2023-004209 filed in Japan on Jan. 16, 2023 the entire contents of which are hereby incorporated by reference.

### BACKGROUND

This disclosure relates to a sensor system and a method of detecting a target substance with a sensing element.

Chemical sensors for detecting a specific kind of chemical substance and the concentration thereof have been developed; for example, chemical sensors for detecting leakage of hydrogen gas have been developed. Although a plurality types of hydrogen gas sensors different in sensing scheme are known, common types of sensors are required to operate under high temperature for higher response speed or cleaning effect. Such hydrogen gas sensors required to operate under high temperature are demanded to eliminate the possibility of explosion caused by contact of overcurrent or a spark in an electric circuit with hydrogen.

As another type of chemical sensor, hydrogen gas sensors that detect hydrogen gas by an optical scheme are known. For example, the hydrogen gas sensor disclosed in JP 2017-172993 A includes a sensor element having a layered structure including a magnetic layer and a hydrogen gas sensing layer. When light for measurement hits the film surface of the sensing element, the polarization angle of the light rotates because of the magneto-optical effect of the magnetic layer. When the hydrogen gas sensing film is in contact with hydrogen, the optical characteristics of the sensing film change, so that the amount of rotation of the polarization angle changes.

Accordingly, the hydrogen gas sensor measures this change to detect hydrogen. The major components of this hydrogen gas sensor, which are a light source, a photodetector, a sensing element, and a magnetic field applicator, are disposed across the atmosphere to be examined. The optical hydrogen gas sensor does not need to be heated to high temperature or an energizing component disposed in the atmosphere to be examined. Accordingly, the aforementioned problem can be avoided; hydrogen gas leakage can be detected more safely.

Since the hydrogen gas sensor according to JP 2017-172993 A utilizes magneto-optical effect for measurement, magnetic material is a requisite for the configuration of the sensor. In addition to this, a magnetic field application mechanism for controlling magnetization of the magnetic material is also necessary. According to the disclosed film configuration, the layered film includes a hydrogen gas sensing layer, a metallic reflection layer, and a magnetic metal layer. JP 2017-172993 A further discloses a configuration including more layers. The necessity of the magnetic field application mechanism and the complexity of the film configuration of the layered film may lead to a higher cost.

### SUMMARY

A sensor system according to an aspect of this disclosure includes a sensing element, an illumination optical system including a light source, the illumination optical system being configured to obliquely illuminate the sensing element, and a detector device configured to detect light reflected off the sensing element. The sensing element includes a chemical sensing layer configured to change in an optical characteristic in response to contact with a target substance, a reflection layer configured to reflect at least part of incident light, and an intermediate layer located between the reflection layer and the chemical sensing layer. The detector device is configured to separately detect p-polarized light and s-polarized light reflected off the sensing element.

An aspect of this disclosure is a method of detecting a predetermined target substance with a sensing element. The sensing element includes a chemical sensing layer configured to change in an optical characteristic in response to contact with the target substance, a reflection layer configured to reflect at least part of incident light; and an intermediate layer located between the reflection layer and the chemical sensing layer. The method includes illuminating the sensing element obliquely, separately detecting p-polarized light and s-polarized light reflected off the sensing element, and generating a result of detecting whether the target substance exists based on a comparison result of intensities of the p-polarized light and the s-polarized light.

It is to be understood that both the foregoing general description and the following detailed description are exemplary and explanatory and are not restrictive of this disclosure.

### BRIEF DESCRIPTION OF THE DRAWINGS

FIG. 1 schematically illustrates a configuration example of a hydrogen gas sensor system in an embodiment of this specification;

FIG. 2A illustrates a configuration example of a sensing element;

FIG. 2B illustrates a configuration example of a sensing element;

FIG. 3 is a cross-sectional diagram schematically illustrating a configuration example of a layered film of a sensing element in an embodiment of this specification;

FIG. 4 provides simulation results for explaining the principle of detecting hydrogen gas with the sensing element illustrated in FIG. 3;

FIG. 5A provides simulation results on the reflectance for p-polarized light and s-polarized light varied because of reaction of the multilayer sensing film of the sensing element illustrated in FIG. 3 to hydrogen gas;

FIG. 5B provides simulation results on the reflectance for p-polarized light and s-polarized light varied because of reaction of the multilayer sensing film of the sensing element illustrated in FIG. 3 to hydrogen gas;

FIG. 6 provides the ratio (lp/ls) of the intensity lp of reflected p-polarized light to the intensity ls of reflected s-polarized light calculated from the simulation results on p-polarized light and s-polarized light in FIG. 5B;

FIG. 7 schematically illustrates a configuration example of a hydrogen gas sensor system in an embodiment of this specification;

FIG. 8 is a cross-sectional diagram schematically illustrating a configuration example of a sensing element in an embodiment of this specification;

FIG. 9 is a graph of measurement results with the configuration example illustrated in FIGS. 7 and 8;

FIG. 10 is a cross-sectional diagram schematically illustrating a configuration example of a sensing element in an embodiment of this specification;

FIG. 11 is a graph of measurement results with the configuration example illustrated in FIGS. 7 and 10;

FIG. 12A schematically illustrates a configuration example of a hydrogen gas sensor system in an embodiment of this specification;

FIG. 12B schematically illustrates a configuration example of a hydrogen gas sensor system in an embodiment of this specification;

FIG. 13 is a cross-sectional diagram schematically illustrating a configuration example of a sensing element in an embodiment of this specification;

FIG. 14 is a graph of measurement results with the configuration example illustrated in FIGS. 12A and 13;

FIG. 15 schematically illustrates a configuration example of a hydrogen gas sensor system in an embodiment of this specification;

FIG. 16 is a cross-sectional diagram schematically illustrating a configuration example of a sensing element in an embodiment of this specification; and

FIG. 17 provides simulation results with the multilayer film illustrated in FIG. 16.

### EMBODIMENTS

Hereinafter, embodiments of this disclosure will be described with reference to the accompanying drawings. The elements in each drawing are changed in size or scale as appropriate to be well recognized in the drawing. The hatches in the drawings are to distinguish the elements and are not necessarily to represent cross-sections. It should be noted that the embodiments are merely examples to implement this disclosure and not to limit the technical scope of this disclosure.

Embodiments of a sensor system are described. The sensor systems in some embodiments of this specification are optical chemical sensor systems. An optical chemical sensor system detects a target substance by measuring the change in optical characteristics of a sensing element caused by contact with the target substance. The optical chemical sensor systems in the embodiments of this specification obliquely illuminate a sensing element and measure the reflected p-polarized light and s-polarized light.

The sensing element includes a thin film layered structure including a chemical sensing layer, an optical interference layer, and a reflection layer, for example. The optical interference layer is an intermediate layer sandwiched between the chemical sensing layer and the reflection layer. Each of the chemical sensing layer and the reflection layer reflects all or part of a specific wavelength of light. The material and the thickness of each film of the layered structure are determined so that the layered structure will make the light having the wavelength to be used for sensing behave as follows: the light reflected off the reflection layer and the light reflected off the chemical sensing layer interfere with each other to provide p-polarized light or s-polarized light with a condition close to the resonance condition. The incident light to the sensing element hits the layered structure and the light reflected off the layered structure resulting from the above-described interference is detected by a detector device.

The embodiments of this disclosure include an oblique-incidence optical system configured so that the light to be used for detection hits the thin film layered structure of the sensing element obliquely. Some part of the light that obliquely enters the sensing element reflects off the interface on the entrance side of the reflection layer. Some part of the light transmitted through the reflection layer passes through the intermediate layer, reflects off the chemical sensing layer, passes through the intermediate layer and the reflection layer again, interferes with the light reflecting off the reflection layer, and is detected by the detector device.

The polarization components in an oblique-incidence optical system can be defined as p-polarized light and s-polarized light. The effects of the interference occurring at the layered structure onto p-polarized light and s-polarized light are different and therefore, the ratio of the reflected light detected by a detector device to the incident light, namely the reflectance, is observed differently between p-polarized light and s-polarized light.

When the chemical sensing layer contacts a target substance, the optical characteristics of the chemical sensing layer change depending on the concentration of the substance, so that the condition of the layered structure to cause interference changes. As described above, the layered structure is configured to provide a condition closer to the resonance condition for p-polarized light or s-polarized light. Accordingly, slight change in optical characteristics of the chemical sensing layer results in deviation from the resonance condition and causes a significant change in reflectance. This change in reflectance provides a large difference in the light intensity detected by the detector device, enabling detection of the target substance with high sensitivity and high resolution.

The change of the chemical sensing layer appears in the reflectance for both p-polarized light and s-polarized light. If the intensity of light from the light source varies, the variation affects the p-polarized light and the s-polarized light at the same proportions. Therefore, the effect of the variation in intensity of light from the light source can be eliminated by comparing the intensities of detected p-polarized light and s-polarized light. Hence, the target substance can be detected with high sensitivity, high resolution, and high stability.

To be detected by the optical chemical sensor system is the concentration of the target substance; the target substance can be pH, a gas such as hydrogen, oxygen, carbon dioxide, chlorine, or a nitrogen oxide, DNA, or an enzyme. The optical chemical sensor system can be an optical ion sensor system for detecting a pH value, an optical gas sensor system for detecting a gas, or an optical biosensor system for detecting DNA or an enzyme. The following embodiments specifically describe optical hydrogen gas sensor systems by way of example. The optical sensor system may be simply referred to as sensor system.

### Embodiment 1

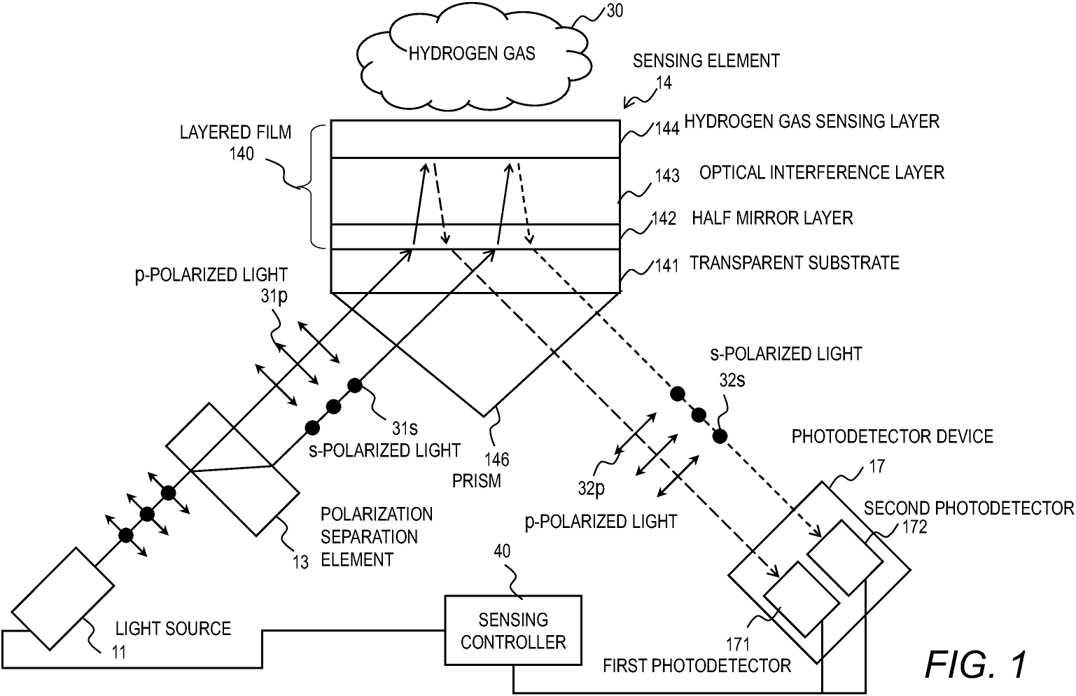

FIG. 1 schematically illustrates a configuration example of a hydrogen gas sensor system in an embodiment of this specification. The hydrogen gas sensor system is an example of an optical chemical sensor system and the target substance to be detected is hydrogen gas. The hydrogen gas sensor system can detect the concentration of hydrogen gas. The lines with an arrow in FIG. 1 represent optical paths. The hydrogen gas sensor is based on an oblique-incidence optical system.

The hydrogen gas sensor system includes a light source 11, a polarization separation element (polarization separator) 13, a sensing element 14, a photodetector device 17, and a sensing controller 40. The illumination optical system includes the light source 11 and the polarization separation element 13. The sensing controller 40 controls the other components of the hydrogen gas sensor system, measures the intensity of light reflected off the sensing element 14, and calculates a measurement value based on the intensity of the reflected light.

The sensing element 14 includes a transparent substrate 141 and a layered film 140 provided on the transparent substrate 141. The layered film 140 includes a half mirror layer 142, an optical interference layer 143, and a hydrogen gas sensing layer 144 stacked in this order on the transparent substrate 141. A prism 146 is attached on the other side of the transparent substrate 141 opposite to the layered film 140 and the prism 146 is optically coupled to the transparent substrate 141 by not-shown immersion oil. The prism 146 reduces the reflection of incident light off the reverse side of the transparent substrate 141 on which the layered film 140 is not provided.

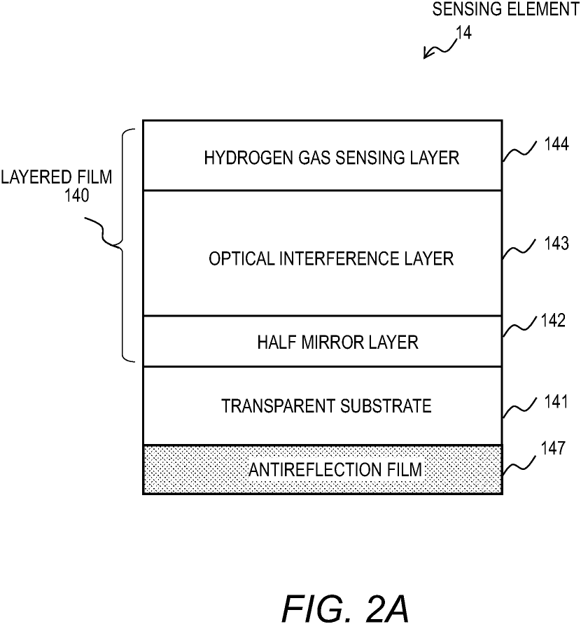

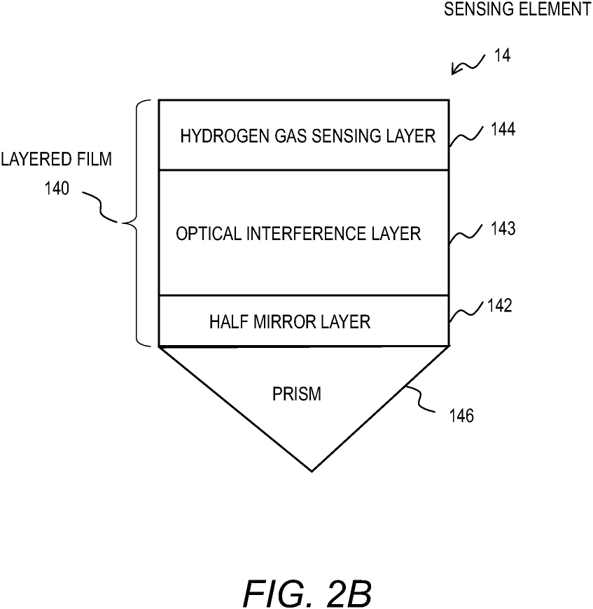

FIG. 2A illustrates another configuration example of the sensing element 14. The configuration example illustrated in FIG. 2A includes an antireflection film 147 provided on the side of the transparent substrate 141 opposite to the layered film 140, in place of the prism 146. The antireflection film 147 can be a multilayered dielectric film, which can be a structure consisting of a thin ZnO film as a high refractive layer and a thin SiO₂ film as a low refractive layer stacked in this order on the transparent substrate 141, for example. The transparent substrate 141 is sandwiched between the antireflection film 147 and the layered film 140. The antireflection film 147 can reduce the reflection of the incident light off the reverse side of the transparent substrate 141 on which the layered film 140 is not provided, like the prism 146.

FIG. 2B illustrates another configuration example of the sensing element 14. The sensing element 14 illustrated in FIG. 2B has a configuration in which the transparent substrate 141 is omitted from the configuration example of the sensing element 14 illustrated in FIG. 1. The layered film 140 is provided directly on a surface of the prism 146. Immersion oil for optical coupling between the transparent substrate 141 and the prism 146 is no longer necessary, thus avoiding contamination of the area around the element by the oil.

Returning to FIG. 1, the surface of the hydrogen gas sensing layer 144 is in contact with hydrogen gas 30 or the target substance. The hydrogen gas sensor system detects hydrogen gas by measuring the variation in optical characteristics of the sensing element 14 caused by the hydrogen gas 30. More specifically, the hydrogen gas sensor system detects the concentration of hydrogen gas by measuring the difference in reflectance of the layered film 140 for p-polarized light and s-polarized light depending on the concentration of hydrogen gas.

In the configuration of FIG. 1, the hydrogen gas sensor system obliquely illuminates the layered film 140 through the prism 146 from behind the reverse side of the transparent substrate 141 on which the layered film 140 for detecting hydrogen gas 30 is not provided. The photodetector device 17 detects the reflected p-polarized light 32p and the reflected s-polarized light 32s, and the sensing controller 40 determines the hydrogen concentration based on the difference between their intensities. The difference between their intensities can be expressed by a function using a delta or a ratio, as will be described later.

The layered film 140 has a structure such that the light reflected off the interface between the half mirror layer 142 and the transparent substrate 141 and the light that has entered the layered film 140 and is reflected off the hydrogen gas sensing layer 144 interfere with each other. For simplicity of illustration, the light reflected off the interface between the half mirror layer 142 and the transparent substrate 141 is not shown in FIG. 1. The same applies to the other drawings. The hydrogen gas sensor system obliquely illuminates the sensing element 14 with the light emitted from the light source 11. An oblique-incidence optical system usually provides different reflectance values for p-polarized light and s-polarized light. Moreover, a layered film like the one in this embodiment imposes different interference conditions onto p-polarized light and s-polarized light.

The behaviors of reflection of p-polarized light and s-polarized light off the layered film 140 providing the above-described conditions appear in the reflectance of the layered film 140 for p-polarized light and s-polarized light. When the proportion of the intensity of p-polarized incident light to the intensity of s-polarized incident light is kept constant, the intensity ratio of reflected light can be calculated by detecting the intensities of the reflected p-polarized light 32p and the reflected s-polarized light 32s with the photodetectors 171 and 172. Using the intensity ratio of the reflected p-polarized light 32p to the reflected s-polarized light 32s eliminates the influence of possible variation in intensity of light emitted from the light source.

The light source 11 emits light to reach the layered film 140 provided on the transparent substrate 141 through the prism 146. The light source 11 can be a monochromatic light source for emitting single-wavelength light, such as a semiconductor laser, an LED, or a gas laser.

The polarization separation element 13 is disposed on the optical path between the light source 11 and the sensing element 14. The polarization separation element 13 separates the light from the light source 11 into p-polarized light 31p and s-polarized light 31s. The p-polarized light 31p and s-polarized light 31s hit the sensing element 14. The p-polarized light 31p and the s-polarized light 31s travel along different optical paths and hit different points of the sensing element 14.

The p-polarized light 31p and s-polarized light 31s separated by the polarization separation element 13 travel along different optical paths, pass through the prism 146, and reach the reverse side of the transparent substrate 141 on which the layered film 140 is not provided. They hit the reverse side of the transparent substrate 141 obliquely with respect to the layering direction of the layered film 140 (the direction normal to the transparent substrate 141). An example of the incident angle is 45°.

The p-polarized light 31 and s-polarized light 31s that have entered the transparent substrate 141 partially reflect off the interface between the transparent substrate 141 and the half mirror layer 142. The remaining light passes through the half mirror layer 142 and the optical interference layer 143, reflects off the chemical sensing layer 144, passes through the optical interference layer 143 and the half mirror layer 142 again in the reverse direction, and interferes with the aforementioned light reflecting off the interface between the transparent substrate 141 and the half mirror layer 142.

On this occasion, the interference conditions for the p-polarized light and the s-polarized light are different. For the wavelength of light from the light source 11 to be used for sensing, the layered film 140 is configured so that the interference to either p-polarized light or s-polarized light will generate a condition close to its resonance condition. The intensity of the reflected polarization component provided with a condition close to the resonance condition will take an extremely small value because of the interference. On the other hand, the intensity of the reflected polarization component provided with a condition off the resonance condition will take a relatively large value.

The p-polarized light 32p and the s-polarized light 32s reflected off the sensing element 14 travel along different optical paths. The reflected p-polarized light 32p and the reflected s-polarized light 32s are detected by a first photodetector 171 and a second photodetector 172, respectively. The first photodetector 171 and the second photodetector 172 are components of the detector device 17 and they are disposed at different locations. The first photodetector 171 is located on the optical path of the reflected p-polarized light 32p to detect the intensity of the reflected p-polarized light 32p and the second photodetector 172 is located on the optical path of the reflected s-polarized light 32s to detect the intensity of the reflected s-polarized light 32s.

The sensing controller 40 controls light emission of the light source 11 and further, receives detection signals from the first photodetector 171 and the second photodetector 172. The sensing controller 40 receives signals representing the intensities of the reflected p-polarized light 32p and the reflected s-polarized light 32s from the first photodetector 171 and the second photodetector 172. The sensing controller 40 calculates the concentration of hydrogen gas based on the result of comparison of those signals.

For example, the sensing controller 40 determines a hydrogen concentration from the intensities of the reflected p-polarized light 32p and the reflected s-polarized light 32s, using a predefined function (including a look-up table). The function can include the ratio of the intensity of p-polarized light to the intensity of s-polarized light as a variable. If the proportion of the p-polarization component to the s-polarization component in the illumination system is kept constant, the variation in intensity of light emitted from the light source 11 can be ignored by using the ratio of the intensity of reflected p-polarized light to the intensity of reflected s-polarized light.

The sensing element 14 includes a layered film 140 consisting of a half mirror layer 142, an optical interference layer 143, and a hydrogen gas sensing layer 144 stacked in this order on the transparent substrate 141, as illustrated in FIG. 1. Each of these layers can be composed of a single layer or a plurality of layers. The hydrogen gas sensing layer 144 is a chemical sensing layer, which is made of material appropriate for the substance to be detected.

The hydrogen gas sensing layer 144 changes in its optical characteristics such as the refractive index and the absorption coefficient in response to contact with hydrogen gas. The hydrogen gas sensor system measures the changes of the intensities of reflected p-polarized light and s-polarized light caused by the change of the optical characteristics of the hydrogen gas sensing layer 144 to detect hydrogen gas.

The optical interference layer 143 is an intermediate layer having a structure to make the light that enters the layered film 140 out of the light illuminating the layered film 140 and reflects off the hydrogen gas sensing layer 144 interfere with the light that reflects off the interface between the transparent substrate 141 and the half mirror layer 142. For example, the value of the sum of the product of the thickness and the refractive index of the half mirror layer 142 and the product of the thickness and the refractive index of the optical interference layer 143 is larger than approximately ¼ of the wavelength of the illumination light.

The half mirror layer 142 reflects some part of the incident light and transmits some other part. The half mirror layer 142 has a thickness that allows the light illuminating the layered film 140 to enter the inside of the layered film 140. For example, the half mirror layer 142 can have a thickness more than 0 nm and not more than 30 nm. The hydrogen gas sensing layer 144 has a thickness enough to reflect the light that enters the inside of the layered film 140; for example, it can have a thickness not less than 20 nm. A thicker hydrogen gas sensing layer 144 can reduce the influence of variation of the surface condition of the sensing layer, enabling more stable detection of the target substance.

As to the material for the hydrogen gas sensing layer 144, any material can be employed that varies in its optical characteristic such as refractive index or absorption coefficient by reacting to hydrogen gas. An example of such material is palladium (Pd) that significantly varies in optical characteristics in response to contact with hydrogen gas. A palladium-containing thin film can be employed as the hydrogen gas sensing layer 144. Such a hydrogen gas sensing layer 144 contributes to provision of a hydrogen gas sensor operable at room temperature and having high sensitivity because palladium has characteristics to absorb and desorb hydrogen gas under room tem perature.

As to the material for the optical interference layer 143, common transparent oxides, transparent nitrides, and transparent fluorides such as silicon dioxide (SiO₂), zinc oxide (ZnO), magnesium oxide (MgO), titanium oxide (TiO₂), aluminum nitride (AlN), silicon nitride (Si₃N₄), and magnesium fluoride (MgF₂) can be listed. The optical interference layer 143 can be made of a dielectric having high transmissivity to the wavelength of light emitted from the light source 11.

As to the material for the half mirror layer 142, common metallic materials including metals such as silver (Ag), aluminum (Al), gold (Au), copper (Cu), and tantalum (Ta) and alloys containing such metals can be listed. The material of the half mirror layer 142 can have a high reflectance for the wavelength of light emitted from the light source 11. The transparent substrate 141 can be a glass substrate having a thickness of approximately 0.5 mm (500 µm), for example.

In an embodiment of this specification, the layered film 140 of the sensing element 14 is composed of non-magnetic materials. Magnetic material is requisite for the layered film of the sensor according to JP 2017-172993 A because the operating principle thereof is to detect a rotation of the plane of polarization caused by magneto-optical effect. However, the layered film 140 in an embodiment of this specification does not need to include magnetic material, although it is not requisite that the layered film 140 is composed of only non-magnetic materials. Materials optimum to attain the characteristics demanded in the embodiment are to be selected, no matter whether the material has magnetic property or not.

In the case of choosing to include a magnetic material, a material having a high magnetic coercive force that does not reverse the magnetization even in the highest magnetic field expected in the use environment and showing a hysteresis loop with high squareness should be selected. Then, the effect of the external magnetic field is suppressed; the layered film can have more stable characteristics.

As described above, the hydrogen gas sensor system illuminates the layered film 140 in a direction oblique to the normal to the layered film 140 and detects the p-polarized light and s-polarized light reflected off the layered film 140. The hydrogen gas sensor system detects hydrogen gas by detecting the change in optical characteristics of the hydrogen gas sensing layer 144 caused by contact with hydrogen gas 30 in the form of optical signals indicating the changes in intensity of p-polarized light and s-polarized light reflected off the layered film 140.

The hydrogen gas sensing layer 144 changes in its optical characteristics in response to contact with hydrogen gas and therefore, the interference condition of the layered film 140 changes. The interference condition changes differently for p-polarized light and s-polarized light; especially, the reflectance for the polarization component provided with a condition close to the resonance condition changes significantly because of the change in optical characteristics of the hydrogen gas sensing layer 144 caused by contact with hydrogen. As a result, the intensity ratio of the reflected p-polarized light to the reflected s-polarized light changes significantly. As understood from this description, the optical signal can be enhanced largely because of the optical interference occurring in the layered film 140, so that hydrogen gas can be detected with high sensitivity. The same applies to other target substances and layered films including a chemical sensing layer therefor.

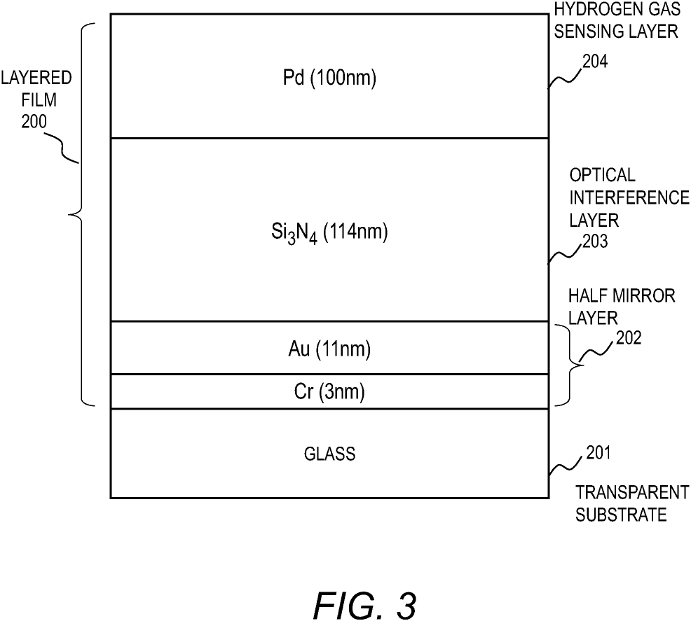

FIG. 3 is a cross-sectional diagram schematically illustrating a configuration example of the layered film (layered sensing film) of a sensing element in an embodiment of this specification. The layered film 200 for sensing hydrogen gas is provided on a transparent glass substrate 201. The layered film 200 consists of a half mirror layer 202, an optical interference layer 203, and a hydrogen gas sensing layer 204.

The half mirror layer 202 consists of a Cr thin film of 3 nm in thickness and an Au thin film of 11 nm in thickness. The optical interference layer 203 is a silicon nitride (Si₃N₄) thin film of 114 nm in thickness. The hydrogen gas sensing layer 204 is a Pd thin film of 100 nm in thickness. The half mirror layer 202, the optical interference layer 203, and the hydrogen gas sensing layer 204 are stacked on the transparent substrate 201 in this order.

An antireflection film or a prism can be provided on the reverse side of the transparent substrate 201 on which the layered film 200 is not provided to reduce the light reflected off the transparent substrate 201.

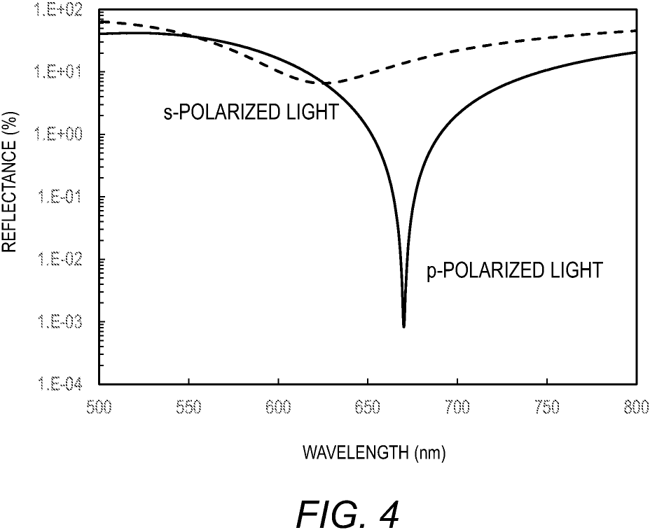

FIG. 4 provides simulation results on the reflection characteristic of the layered sensing film 200 of FIG. 3. In FIG. 4, the horizontal axis represents the wavelength of the light from the light source and the vertical axis represents the reflectance for p-polarized light and s-polarized light. FIG. 4 indicates that the layered sensing film 200 under the condition of film thicknesses illustrated in FIG. 3 provides a resonance condition to p-polarized light when the wavelength is 670 nm. The vertical axis is a logarithmic axis; the reflectance varies significantly around the resonance condition and in principle, it becomes 0 under the resonance condition. The reflectance for s-polarized light under the same condition is several digits higher than the reflectance for p-polarized light and the variation is small. Specifically, in FIG. 4, at a wavelength of 670 nm, the reflectance of s-polarized light is about 14 % and that of p-polarized light is about 0.0008 %. Therefore, when the ratio of reflectance is calculated, the reflectance of s-polarized light is 4 orders of magnitude higher than that of p-polarized light (about 17500 times higher).

Although the layered sensing film 200 provides a resonance condition for s-polarized light, the wavelength is different and the variation in reflectance around the resonance condition is gentle, compared to the one for p-polarized light. Although this section has described a design that provides a steep resonance condition to p-polarized light, a layered film that provides a steep resonance condition to s-polarized light is available, depending on the design of the layered film. Although this section has provided an example where the horizontal axis represents the wavelength, a different parameter that affects the interference condition, such as the incident angle or an optical characteristic of the layered film, can be employed for the horizontal axis. Even in that case, similar response can be acquired around the resonance condition.

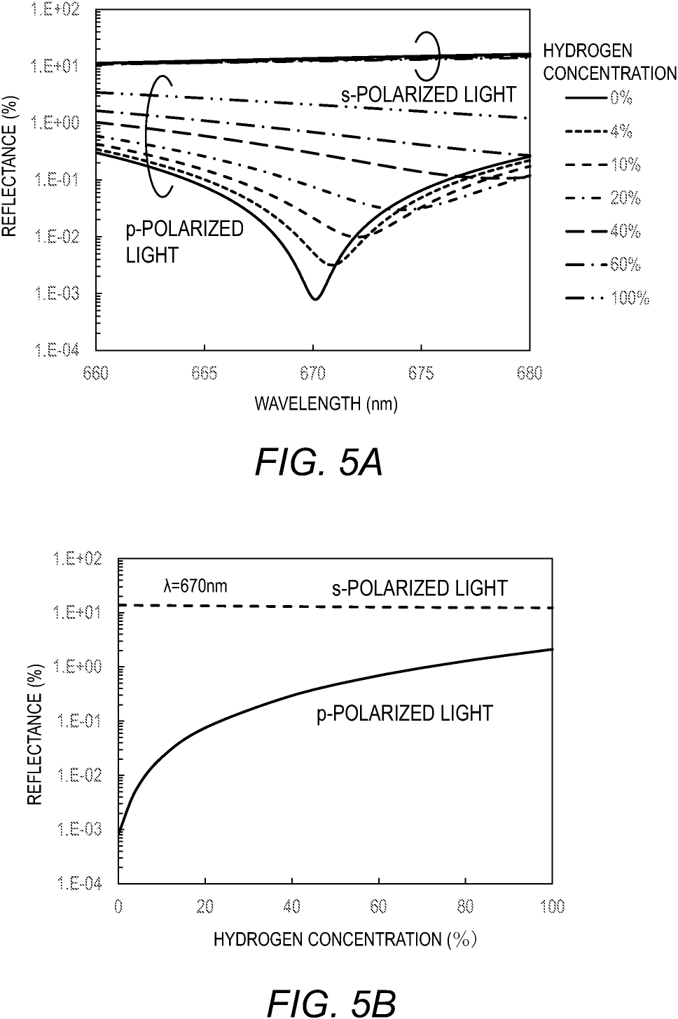

FIGS. 5A and 5B provide simulation results on the reflection characteristic of the layered sensing film 200 illustrated in FIG. 3, specifically, the variation in the characteristic depending on the concentration of hydrogen in contact with the hydrogen gas sensing layer. FIG. 5A provides simulation results on the wavelength dependency of the reflectance in FIG. 4 when the concentration of the hydrogen in contact with the hydrogen gas sensing layer is changed. In FIG. 5A, the horizontal axis represents the wavelength of the light from the light source and the vertical axis represents the reflectance for p-polarized light and s-polarized light. As described with reference to FIG. 4, the layered sensing film 200 provides a resonance condition to 670-nm p-polarized light when the hydrogen concentration is 0%. As for the p-polarized light, the resonance condition shifts toward a longer wavelength and the variation in reflectance around the resonance condition becomes gentler as the hydrogen concentration is raised.

On the other hand, as for the s-polarized light, the overall reflectance within this graph slightly reduces as the hydrogen concentration is raised. When seeing the results at a fixed wavelength of 670 nm where p-polarized light is provided with a resonance condition, the reflectance for p-polarized light increases as the hydrogen concentration is raised. However, the reflectance for s-polarized light is originally high; it varies with hydrogen concentration but the variation is very little in this graph whose vertical axis is a logarithmic axis. Although the vertical axis of FIG. 5A represents reflectance, the detected actually is the intensity of reflected light.

The element designed to have the characteristics as shown in FIG. 5A enables determining the hydrogen concentration of an atmosphere to which the hydrogen sensing film is exposed from the combination of the reflectance for s-polarized light and the reflectance for p-polarized light. FIG. 5B provides data at the wavelength of 670 nm extracted from FIG. 5A. The horizontal axis represents hydrogen concentration and the vertical axis is a logarithmic axis representing the reflectance of the layered sensing film 200 for p-polarized light and s-polarized light. The solid line represents the variation of the reflectance for p-polarized light with respect to the hydrogen concentration and the dashed line represents the variation of the reflectance for s-polarized light with respect to the hydrogen concentration.

According to the simulation results in FIG. 5B, the reflectance for p-polarized light changed by approximately three orders of magnitude when the hydrogen concentration was changed from 0% to 100%. However, the reflectance for s-polarized light changed very little when the hydrogen concentration was changed from 0% to 100%; the variation is small, compared to the reflectance for p-polarized light. Accordingly, assuming that the intensity of reflected s-polarized light with this configuration example is constant, the hydrogen concentration can be determined from only the intensity of reflected p-polarized light. However, even in the case of using this example, measuring the intensities of reflected p-polarized light and s-polarized light and using their ratio can eliminate the influence of the variation in intensity of the light from the light source and the minute variation in intensity of the reflected s-polarized light, enabling highly accurate hydrogen gas detection. The reflectance for p-polarized light and the reflectance for s-polarized light can be adjusted by adjusting the material and the thickness of each layer of the layered film.

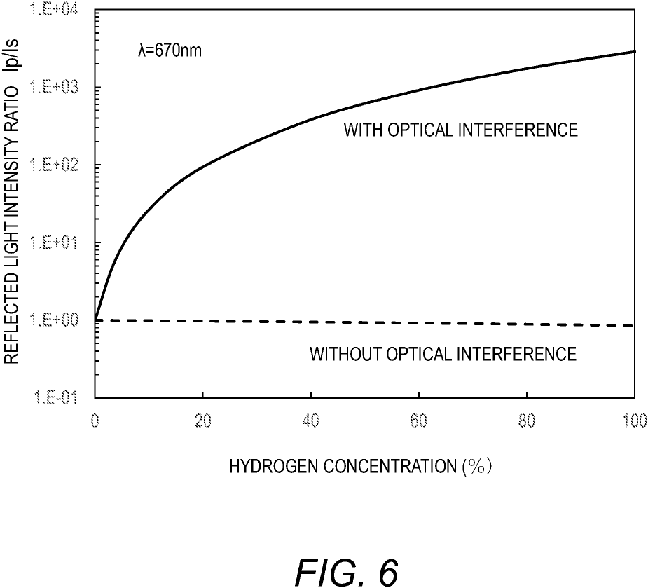

FIG. 6 provides the ratio (lp/ls) of the intensity lp of reflected p-polarized light to the intensity ls of reflected s-polarized light calculated from the simulation results on p-polarized light and s-polarized light in FIG. 5B acquired from a layered film, in comparison to the ratio acquired from a single layer film. The horizontal axis represents hydrogen concentration. The vertical axis is a logarithmic axis representing the ratio (lp/ls) of the intensity lp of reflected p-polarized light to the intensity ls of reflected s-polarized light. The vertical axis is normalized so that the intensity ratio (lp/ls) takes a value 1 when the hydrogen concentration is 0%. The dashed line represents the variation of the intensity ratio (lp/ls) with respect to the hydrogen concentration in the case of a single Pd layer (100 nm) without a half mirror layer and an optical interference layer. The solid line represents the variation of the intensity ratio (lp/ls) with respect to the hydrogen concentration in the case of the layered film 200 illustrated in FIG. 3.

The calculation result in FIG. 6 indicates that the layered film 200 having optical interference effect sensitively responds to variation of hydrogen concentration. Although the Pd single layer film (100 nm) also exhibits variation in reflection characteristic in response to contact with hydrogen, the variation is very small, compared to the layered film 200 having optical interference effect.

### Embodiment 2

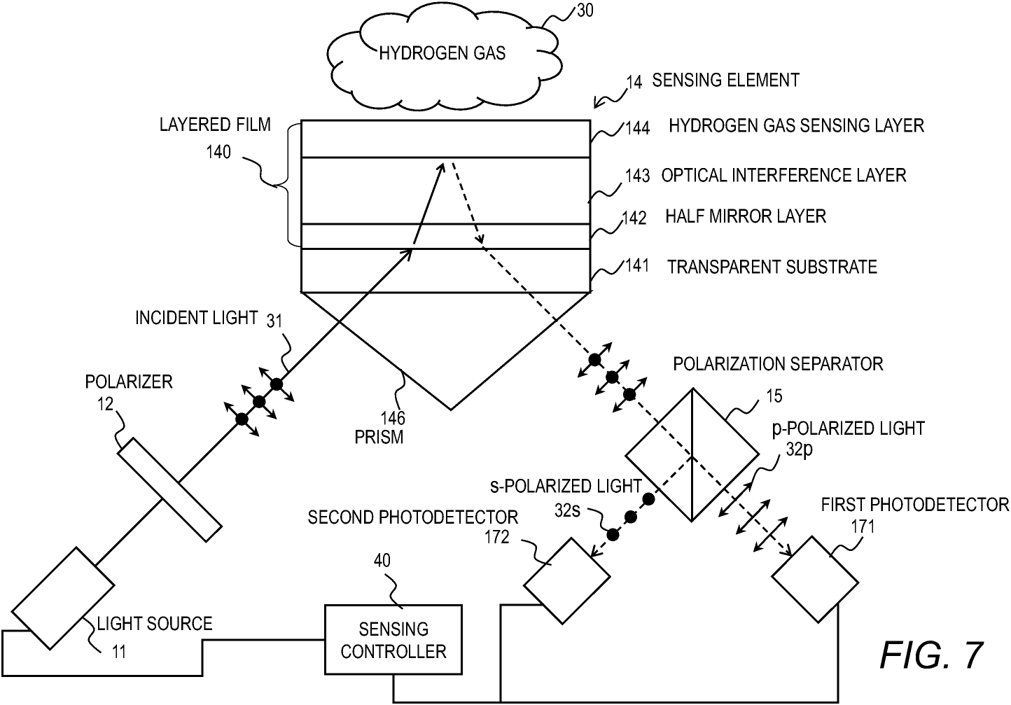

FIG. 7 schematically illustrates a configuration example of a hydrogen gas sensor system in an embodiment of this specification. The following mainly describes differences from the configuration example in FIG. 1. Like the configuration example in FIG. 1, this hydrogen gas sensor system is based on an oblique-incidence optical system; it obliquely illuminates the reverse side of a transparent substrate 141 on which a layered film 140 for sensing hydrogen gas is not provided through a prism 146 to detect hydrogen gas. The sensing element 14 has the same structure as the one in the configuration example in FIG. 1.

The illumination optical system includes a light source 11 and a polarizer 12. The polarizer 12 is disposed on the optical path of the incident light 31 between the light source 11 and the sensing element 14. The polarizer 12 transmits light polarized linearly in a specific direction and attenuates light polarized in the other directions. The polarizer 12 can be adjusted in advance so that the difference in intensity between reflected p-polarized light and reflected s-polarized light within the measurement limit of hydrogen concentration will fall within a predetermined range. For example, the rotation angle of the polarizer 12 can be adjusted so that the intensities of reflected p-polarized light and reflected s-polarized light will be substantially equal when hydrogen gas does not exist. Such appropriate adjustment of the rotation angle of the polarizer 12 increases the accuracy, although the polarizer 12 is optional.

Further, a polarization separator 15 is disposed on the optical path between the sensing element 14 and a photodetector device. Although FIG. 7 does not show a frame indicating the photodetector device 17 shown in FIG. 1 for simplicity of illustration, the photodetector device 17 in FIG. 7 includes a first photodetector 171 and a second photodetector 172, like the photodetector device 17 in the configuration example in FIG. 1. The polarization separator 15 separates p-polarized light 32p and s-polarized light 32s from the light reflected off the sensing element 14. The p-polarized light 32p and s-polarized light 32s from the polarization separator 15 travel along different optical paths.

The linearly polarized light 31 transmitted through the polarizer 12 passes through the prism 146 and hits the layered film 140 of the sensing element 14 with an intensity ratio of the p-polarized component to the s-polarized component in accordance with the polarization angle. As described above, the linearly polarized light 31 hits the layered film 140 at an angle inclined with respect to the normal to the layered film 140. Unlike Embodiment 1, the incident light is not separated to p-polarized light and s-polarized light and the linearly polarized light 31 hits one point of the layered film 140. For this reason, the influence of the in-plane difference in characteristics of the layered film 140 can be avoided.

As described in Embodiment 1, the p-polarized light and the s-polarized light in the incident light are provided with different interference conditions from the layered film 140. Further, the hydrogen gas 30 changes the optical characteristics of the hydrogen gas sensing layer 144. Accordingly, the reflectance of the hydrogen gas sensing layer 144 for p-polarized light and s-polarized light and the interference conditions for p-polarized light and s-polarized light from the layered film 140 change. As a result, the intensity ratio of p-polarized light to s-polarized light in the light reflected off the layered film 140 changes significantly.

The light reflected off the layered film 140 enters the polarization separator 15 and is separated into p-polarized light 32p and s-polarized light 32s. These travel along different optical paths. The first photodetector 171 receives the p-polarized light 32p and outputs its intensity to the sensing controller 40. The second photodetector 172 receives the s-polarized light 32s and outputs its intensity to the sensing controller 40. This configuration of separating the light reflected off the sensing element 14 into p-polarized light and s-polarized light, detecting their intensities, and using their ratio can eliminate the influence of the variation in intensity of light from the light source.

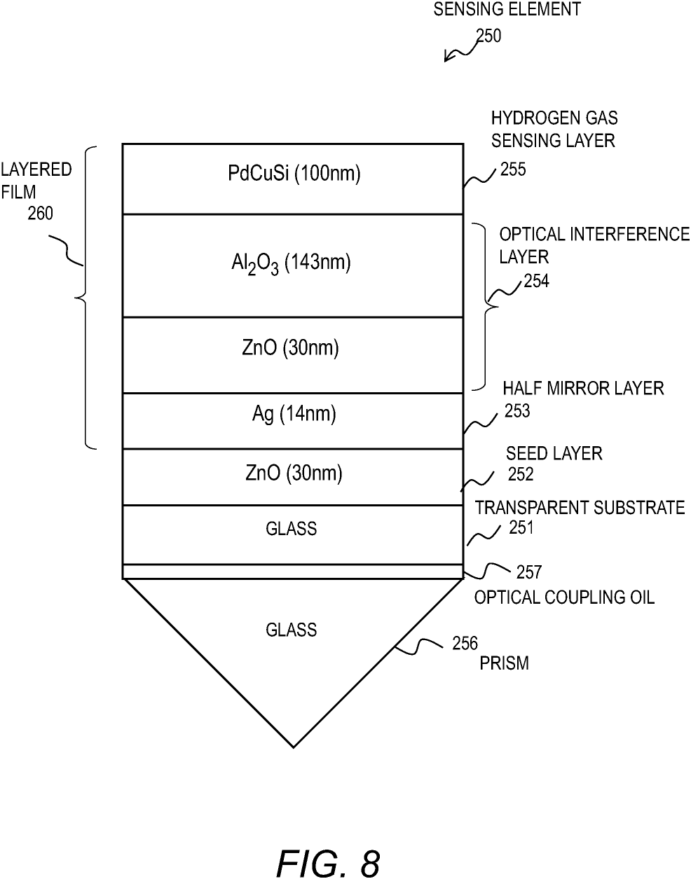

FIG. 8 is a cross-sectional diagram schematically illustrating a configuration example of a sensing element 250 in an embodiment of this specification. The sensing element 250 includes a layered film 260 for sensing hydrogen gas on a transparent glass substrate 251. The layered film 260 consists of a half mirror layer 253, an optical interference layer 254, and a hydrogen gas sensing layer 255. The half mirror layer 253, the optical interference layer 254, and the hydrogen gas sensing layer 255 are stacked on the transparent substrate 251 in this order.

The half mirror layer 253 is an Ag thin film of 14 nm in thickness. The optical interference layer 254 is a layered film consisting of a ZnO thin film of 30 nm in thickness and an Al₂O₃ thin film of 143 nm in thickness. The hydrogen gas sensing layer 255 is a PdCuSi alloy thin film of 100 nm in thickness.

A seed layer 252 is interposed between the layered film 260 and the transparent substrate 251. The seed layer 252 is a ZnO thin film of 30 nm in thickness. The seed layer 252 reinforces the adhesion strength between the layered film 260 and the transparent substrate 251.

A prism 256 is provided on the reverse side of the transparent substrate 251 on which the layered film 260 is not provided and the prism 256 is optically coupled to the transparent substrate 251 with optical coupling oil 257. The prism 256 reduces the reflection of light off the reverse side of the transparent substrate 251. The optical coupling oil 257 reduces the reflection of light off the interface between the transparent substrate 251 and the prism 256.

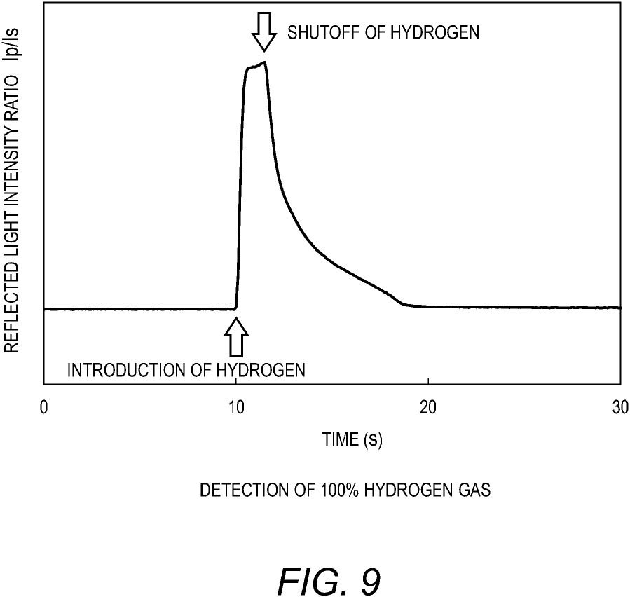

FIG. 9 is a graph of measurement results with the configuration example illustrated in FIGS. 7 and 8. The horizontal axis represents time and the vertical axis represents the ratio (lp/ls) of the intensity lp of reflected p-polarized light and the intensity ls of reflected s-polarized light. In the measurement, the polarizing angle (the rotation angle of the polarizer 12) for the incident light was adjusted so that the intensities of reflected p-polarized light and reflected s-polarized light become equal when the hydrogen concentration is 0%.

The solid line represents the variation in the ratio (lp/ls) of the intensity of p-polarized light to the intensity of s-polarized light in response to introduction and shutoff of hydrogen gas. In the measurement, 100% hydrogen gas was introduced and subsequently the hydrogen gas was shut off. In response to the introduction of hydrogen, the reflected light intensity ratio (lp/ls) elevated largely. After shutting off the hydrogen, the reflected light intensity ratio (lp/ls) fell gradually. This measurement revealed that the concentration of hydrogen can be detected with high sensitivity with the configuration of this embodiment.

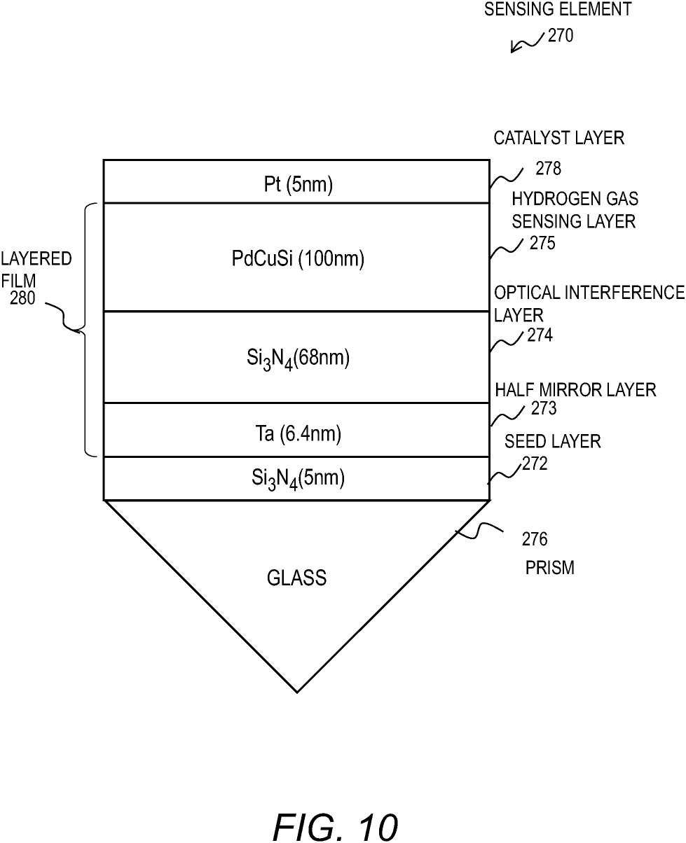

FIG. 10 is a cross-sectional diagram schematically illustrating a configuration example of a sensing element 270 in an embodiment of this specification. The sensing element 270 includes a layered film 280 for sensing hydrogen gas on a prism 276 made of glass. The layered film 280 consists of a half mirror layer 273, an optical interference layer 274, and a hydrogen gas sensing layer 275. The half mirror layer 273, the optical interference layer 274, and the hydrogen gas sensing layer 275 are stacked on the prism 276 in this order.

The half mirror layer 273 is a Ta thin film of 6.4 nm in thickness. The optical interference layer 274 is a Si₃N₄ thin film of 68 nm in thickness. The hydrogen gas sensing layer 275 is a PdCuSi alloy thin film of 100 nm in thickness.

A seed layer 272 is interposed between the layered film 280 and the prism 276. The seed layer 272 is an Si₃N₄ thin film of 5 nm in thickness. The seed layer 272 reinforces the adhesion strength between the layered film 280 and the prism 276.

A catalyst layer 278 is provided to cover the hydrogen gas sensing layer 275. The catalyst layer 278 is a platinum (Pt) thin film of 5 nm in thickness formed on the layered film 280 on the opposite side of the prism 276. The catalyst layer 278 is in contact with the hydrogen gas sensing layer 275. When in contact with hydrogen gas, the catalyst layer 278 divides hydrogen molecules into hydrogen atoms. The hydrogen gas sensing layer 275 changes in its optical characteristics in accordance with the concentration of hydrogen atoms generated by the catalyst layer 278.

Considering applications that are constantly exposed to high concentration hydrogen, the long-term stability of the sensing element can be improved by preventing unnecessary reactions with hydrogen and layers other than the hydrogen gas sensing layer. Among transparent oxides that are candidates for optical interference layer materials, there are some materials that are more stable in hydrogen compounds (hydroxides) formed in the reaction with hydrogen. In selecting an optical interference layer material, using the idea that the optical interference layer material is more stable than hydrogen compounds of the elements constituting the interference layer, the reaction between the optical interference layer and hydrogen can be suppressed and the long-term stability of the sensing element can be improved. An example is silicon nitride. In this embodiment, silicon nitride is used as the interference layer, as explained with reference to FIG. 10.

When the optical interference layer is made of silicon nitride, several metallic materials were tried as half mirror materials, but the adhesion strength of the interface between the optical interference layer and the half mirror layer was weak in some cases. If the adhesion strength of the interface is weak, the interface is not maintained stably in long-term use, and the reproducibility and reliability of the measurement results are reduced. In studying the combination with silicon nitride, we have found that the adhesion strength of the interface is sufficiently high when tantalum (Ta) is used for the half mirror. In an embodiment of this specification, Ta thin film is used as the half mirror material in combination with silicon nitride to make the sensing element more stable.

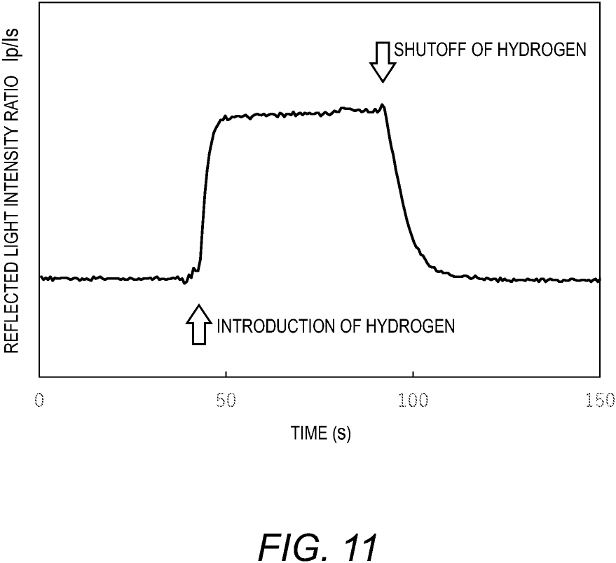

FIG. 11 is a graph of measurement results with the configuration example illustrated in FIGS. 7 and 10. The horizontal axis represents time and the vertical axis represents the ratio (lp/ls) of the intensity lp of reflected p-polarized light and the intensity ls of reflected s-polarized light. In the measurement, the polarizing angle (the rotation angle of the polarizer 12) for the incident light was adjusted so that the intensities of reflected p-polarized light and reflected s-polarized light become equal when the hydrogen concentration is 0%.

The solid line represents the variation in the ratio (lp/ls) of the intensity of p-polarized light to the intensity of s-polarized light in response to introduction and shutoff of hydrogen gas. In the measurement, 100% hydrogen gas was introduced and subsequently the hydrogen gas was shut off. In response to the introduction of hydrogen, the reflected light intensity ratio (lp/ls) elevated largely. After shutting off the hydrogen, the reflected light intensity ratio (lp/ls) fell gradually. This measurement revealed that the concentration of hydrogen can be detected with high sensitivity with the configuration of this embodiment.

### Embodiment 3

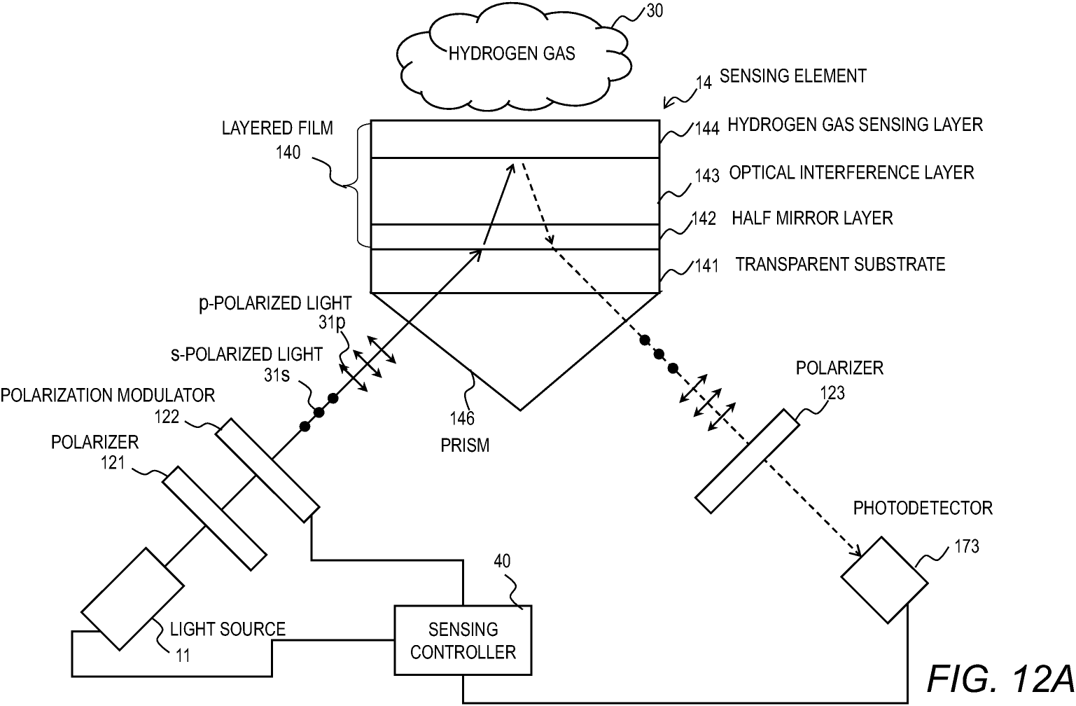

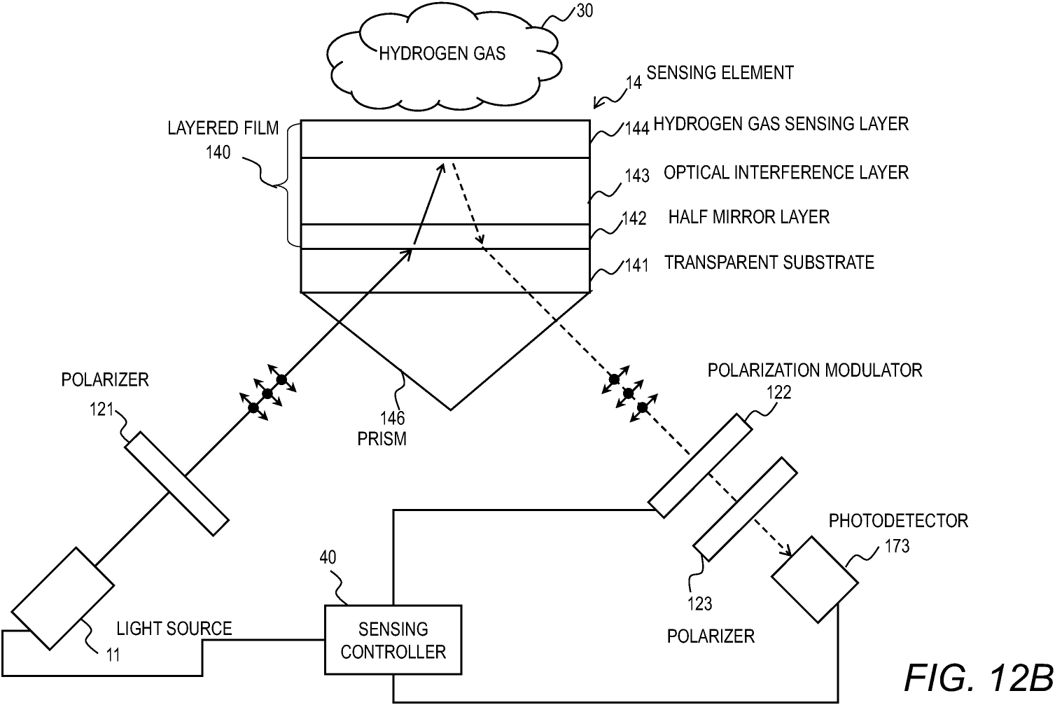

FIG. 12A schematically illustrates a configuration example of a hydrogen gas sensor system in an embodiment of this specification. The following mainly describes differences from the configuration example in FIG. 1. Like the configuration example in FIG. 1, this hydrogen gas sensor system is based on an oblique-incidence optical system; it obliquely illuminates the reverse side of a transparent substrate 141 on which a layered film 140 for sensing hydrogen gas is not provided through a prism 146 to detect hydrogen gas. The sensing element 14 has the same structure as the one in the configuration example in FIG. 1.

The hydrogen gas sensor system in FIG. 12A includes a polarization modulator 122 in the illumination system. The polarization modulator 122 can switch polarization states of the incident light by time-division control. As a result, detection of reflected p-polarized light and reflected s-polarized light with one photodetector is available.

As illustrated in FIG. 12A, the illumination optical system includes a light source 11, a polarizer 121, and the polarization modulator 122. The polarizer 121 is disposed on the optical path of the incident light between the light source 11 and the sensing element 14. The polarizer 121 transmits light polarized linearly in a specific direction and attenuates light polarized in the other directions.

Furthermore, the polarization modulator 122 is disposed on the optical path of the incident light between the polarizer 121 and the sensing element 14. The polarization modulator 122 switches polarizing directions of the linearly polarized light from the polarizer 121. The polarization modulator 122 can utilize liquid crystal, for example. The polarization modulator 122 can modulate the linearly polarized light from the polarizer 121 into either of two kinds of linearly polarized light orthogonal to each other corresponding to p-polarized light and s-polarized light by being controlled.

Another polarizer 123 is disposed on the optical path between the sensing element 14 and the photodetector device. The polarizer 123 can be adjusted in advance so that the difference in intensity between reflected p-polarized light and reflected s-polarized light within the measurement limit of hydrogen concentration will fall within a predetermined range. For example, the rotation angle of the polarizer 123 can be adjusted so that the intensities of reflected p-polarized light and reflected s-polarized light will be substantially equal when hydrogen gas does not exist. Such appropriate adjustment of the rotation angle of the polarizer 123 increases the accuracy, although the polarizer 123 is optional.

The photodetector device includes one photodetector 173. This means that the single photodetector 173 receives reflected p-polarized light and reflected s-polarized light by time division and sends their intensities to the sensing controller 40.

The linearly polarized light transmitted through the polarizer 121 enters the polarization modulator 122. The sensing controller 40 controls the polarization modulator 122 to modulate the linearly polarized light from the polarizer 121 to s-polarized light 31s or p-polarized light 31p. For example, the polarization modulator 122 outputs p-polarized light 31p during a first period and outputs s-polarized light 31s during a second period after the first period.

The p-polarized light 31p and s-polarized light 31s hit the layered film 140 of the sensing element 14 through a prism 146. The p-polarized light 31p and s-polarized light 31s hit the layered film 140 at an angle inclined with respect to the normal to the layered film 140. The p-polarized light 31p and s-polarized light 31s hit the same one point of the layered film 140. For this reason, the influence of the in-plane difference in characteristics of the layered film 140 can be avoided.

As described in Embodiment 1, the p-polarized light and the s-polarized light are provided with different interference conditions from the layered film 140. Further, the hydrogen gas 30 changes the optical characteristics of the hydrogen gas sensing layer 144. Accordingly, the reflectance of the hydrogen gas sensing layer 144 for p-polarized light 31p and s-polarized light 31s and the interference conditions for p-polarized light 31p and s-polarized light 31s from the layered film 140 change. As a result, the intensity ratio of p-polarized light to s-polarized light in the light reflected off the layered film 140 changes significantly.

The p-polarized light reflected off the layered film 140 and the s-polarized light reflected off the layered film 140 enter the polarizer 123 in different periods. Light polarized linearly at a specific polarization angle passes through the polarizer 123 and enters the photodetector 173. The photodetector 173 receives reflected p-polarized light and reflected s-polarized light in different periods and outputs their intensities to the sensing controller 40. This configuration that detects p-polarized light and s-polarized light in different periods enables a single photodetector to detect the intensities of reflected p-polarized light and s-polarized light to be used to detect a hydrogen concentration.

Although FIG. 12A provides a configuration where the polarization modulator 122 is disposed in the illumination system, the polarization modulator 122 can be disposed in the detection system as illustrated in FIG. 12B. That is to say, the polarization modulator 122 can be disposed on the optical path of the reflected light between the sensing element 14 and the polarizer 123. In this case, the rotation angle of the polarizer 121 is adjusted so that the proportion of the intensity of p-polarized light to the intensity of s-polarized light will be constant and the sensing element 14 reflects light containing both components of p-polarized light and s-polarized light.

The polarization modulator 122 rotates the polarization axis of the reflected light by 90 degrees by time-division control. The polarizer 123 is adjusted to transmit only either p-polarized light or s-polarized light. Then, one polarization component passes in the first period and the other polarization component rotated by 90 degrees passes in the subsequent second period. Hence, the same function as in the case where the polarization modulator 122 is disposed in the illumination system is attained.

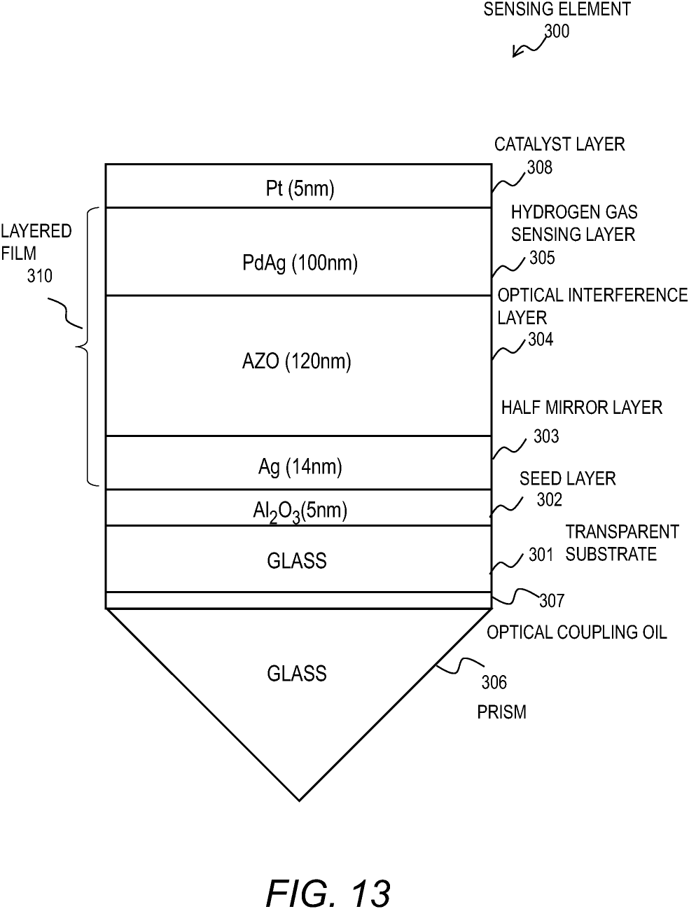

FIG. 13 is a cross-sectional diagram schematically illustrating a configuration example of a sensing element 300 in an embodiment of this specification. The sensing element 300 includes a layered film 310 for sensing hydrogen gas on a transparent glass substrate 301. The layered film 310 consists of a half mirror layer 303, an optical interference layer 304, and a hydrogen gas sensing layer 305. The half mirror layer 303, the optical interference layer 304, and the hydrogen gas sensing layer 305 are stacked on the transparent substrate 301 in this order.

The half mirror layer 303 is an Ag thin film of 14 nm in thickness. The optical interference layer 304 is an aluminum-doped zinc oxide (AZO) thin film of 120 nm in thickness. The hydrogen gas sensing layer 305 is a PdAg alloy thin film of 100 nm in thickness.

A seed layer 302 is interposed between the layered film 310 and the transparent substrate 301. The seed layer 302 is an Al₂O₃ thin film of 5 nm in thickness. The seed layer 302 reinforces the adhesion strength between the layered film 310 and the transparent substrate 301.

A catalyst layer 308 is provided to cover the hydrogen gas sensing layer 305. The catalyst layer 308 is a platinum (Pt) thin film of 5 nm in thickness formed on the layered film 310 on the opposite side of the transparent substrate 301. The catalyst layer 308 is in contact with the hydrogen gas sensing layer 305. When in contact with hydrogen gas, the catalyst layer 308 divides hydrogen molecules into hydrogen atoms. The hydrogen gas sensing layer 305 changes in its optical characteristics in accordance with the concentration of hydrogen atoms generated by the catalyst layer 308.

A prism 306 is provided on the reverse side of the transparent substrate 301 on which the layered film 310 is not provided and the prism 306 is optically coupled to the transparent substrate 301 with optical coupling oil 307. The prism 306 reduces the reflection of light off the reverse side of the transparent substrate 301. The optical coupling oil 307 reduces the reflection of light off the interface between the transparent substrate 301 and the prism 306.

The polarization modulator 122 has a configuration including liquid crystal sandwiched between two glass plates. The side facing liquid crystal of each glass plate is coated with a transparent electrode made of indium-tin-oxide (ITO). The polarization modulation element in this embodiment includes liquid crystal layer of approximately 3.5 µm in thickness. However, the thickness of liquid crystal layer can be selected optimally in view of the optical anisotropy of the liquid crystal, which will be described in the following.

The polarization modulation element employed for this embodiment includes liquid crystal having an optical anisotropy of approximately 0.2 and a thickness of 3.5 µm. This configuration attains a phase shift of 0.7 µm at maximum and therefore, in the case where the wavelength of the light from the light source is 0.63 µm, a phase difference of a half wavelength of 0.315 µm can be attained by application of voltage not more than 10 V. The side facing the liquid crystal of each glass substrate is covered with a polyimide film and the surface of the polyimide film is rubbed in one direction so that the liquid crystal molecules will be aligned uniformly in one direction.

The polarization modulator utilizing liquid crystal configured as described above provides a phase difference to light polarized in two directions orthogonal to each other in transmitting the polarized light therethrough. The phase difference can be controlled with the applied voltage. That is to say, this polarization modulator works as an optical phase plate that varies in the amount of phase shift in accordance with the applied external voltage; it can convert the linearly polarized light from the polarizer 121 into elliptically polarized light, circularly polarized light, or linearly polarized light. This embodiment uses the polarization modulator to switch polarization axes of linearly polarized light.

For example, in the case where the incident light is linearly polarized light having a polarization axis of π/4 with respect to the axis of the polarization modulator, application of a voltage to make a phase difference of an integer multiple of the wavelength of the light of the light source in the first period results in zero phase difference. Accordingly, the polarization modulator outputs linearly polarized light having the same polarization axis as the incident light. In contrast, application of a voltage to make a phase difference of an integer multiple of the wavelength of light of the light source plus a half wavelength has the same effect as a half-wave plate and therefore, the polarization modulator outputs linearly polarized light having a polarization axis rotated by π/2 with respect to the incident linearly polarized light. Although this embodiment changes the direction of polarization with a polarization modulation element, a 90-degree twisted nematic cell can exhibit the same effect.

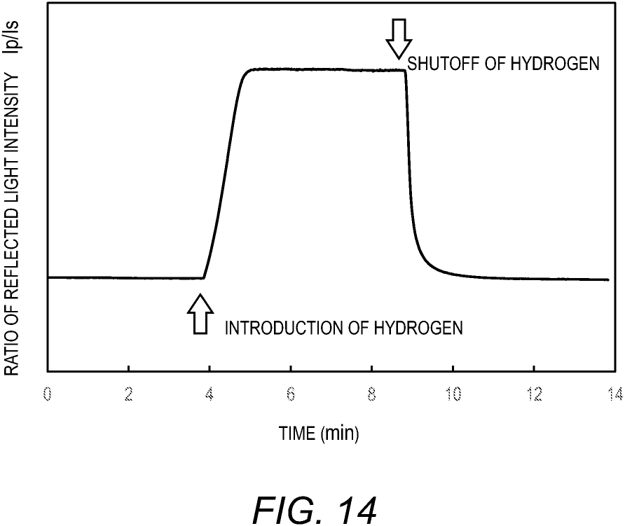

FIG. 14 is a graph of measurement results with the configuration example illustrated in FIGS. 12A and 13. The horizontal axis represents time and the vertical axis represents the ratio (lp/ls) of the intensity lp of reflected p-polarized light and the intensity ls of reflected s-polarized light. In the measurement, the rotation angle of the polarizer 123 was adjusted so that the intensities of reflected p-polarized light and reflected s-polarized light become equal when the hydrogen concentration is 0%.

The solid line represents the variation in the ratio (lp/ls) of the intensity of p-polarized light to the intensity of s-polarized light in response to introduction and shutoff of hydrogen gas. In the measurement, hydrogen and nitrogen mixture gas was introduced, in which the hydrogen concentration was 4%, and subsequently the mixture gas was shut off. In response to the introduction of hydrogen, the reflected light intensity ratio (lp/ls) elevated largely. After shutting off the hydrogen, the reflected light intensity ratio (lp/ls) fell gradually. This measurement revealed that the concentration of hydrogen can be detected with high sensitivity with the configuration of this embodiment.

### Embodiment 4

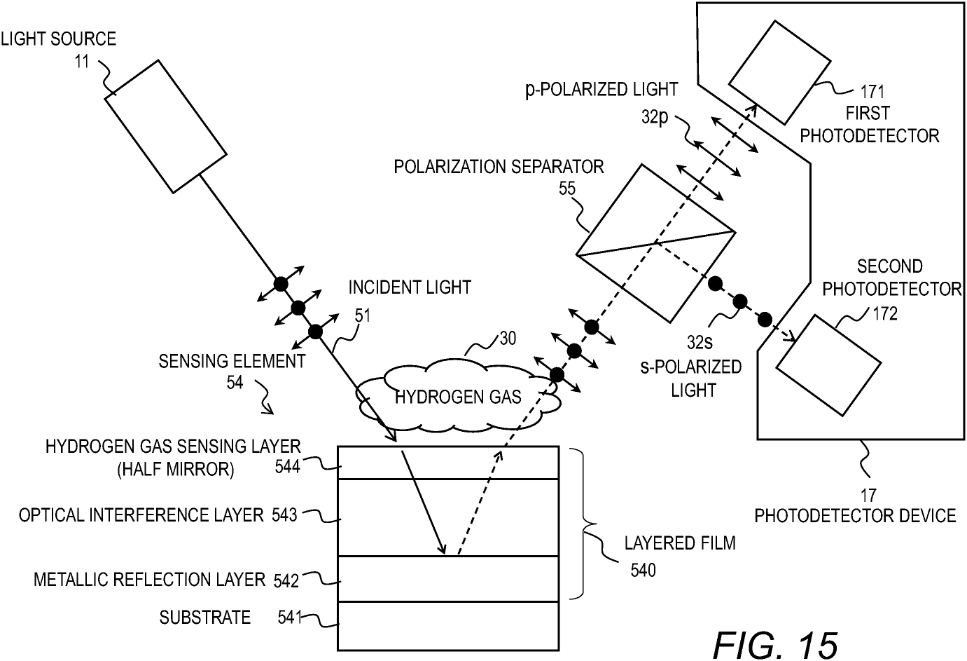

FIG. 15 illustrates a configuration example of a hydrogen gas sensor system in an embodiment of this specification. The following mainly describes differences from the configuration example in FIG. 1. Like the configuration example in FIG. 1, this hydrogen gas sensor system is based on an oblique-incidence optical system. Unlike the configuration example in FIG. 1, the configuration example in FIG. 15 directly and obliquely illuminates the layered film for sensing hydrogen gas to detect the hydrogen gas 30. The illumination optical system includes a light source 11.

A sensing element 54 includes a layered film 540 on a substrate 541. The layered film 540 includes a metallic reflection layer 542, an optical interference layer 543 and a hydrogen gas sensing layer 544 stacked on the substrate 541 in this order. The metallic reflection layer 542 totally reflects light for detecting hydrogen gas. The hydrogen gas sensing layer 544 is a half mirror; it transmits some part of the light for detecting hydrogen gas and reflects some other part.

The light from the light source 11 hits the layered film 540 in the sensing element 54 from above the hydrogen sensing layer 544. As described above, the light hits the layered film 540 at an angle inclined with respect to the direction normal to the layered film 540. The incident light is not separated into p-polarized light and s-polarized light and hits one point. For this reason, the influence of the in-plane difference in characteristics of the layered film 540 can be avoided.

As described in Embodiment 1, the p-polarized light and the s-polarized light in the incident light are provided with different interference conditions from the layered film 540. Further, the hydrogen gas 30 changes the optical characteristics of the hydrogen gas sensing layer 544. Accordingly, the reflectance of the hydrogen gas sensing layer 544 for p-polarized light and s-polarized light and the interference conditions for p-polarized light and s-polarized light from the layered film 540 change. As a result, the intensity ratio of p-polarized light to s-polarized light in the light reflected off the layered film 540 changes significantly.

A polarization separator 55 is disposed on the optical path between the sensing element 54 and a photodetector device 17. The photodetector device 17 includes a first photodetector 171 and a second photodetector 172. The polarization separator 55 separates the light reflected off the sensing element 54 into p-polarized light 32p and s-polarized light 32s. The p-polarized light 32p and s-polarized light 32s from the polarization separator 55 travel along different optical paths.

The first photodetector 171 receives the p-polarized light 32p and outputs the intensity of the p-polarized light 32p to the sensing controller 40. The second photodetector 172 receives the s-polarized light 32s and outputs the intensity of the s-polarized light 32s to the sensing controller 40. This configuration of separating the light reflected off the sensing element 54 into p-polarized light and s-polarized light, detecting their intensities, and using their ratio can eliminate the influence of the variation in intensity of light from the light source.

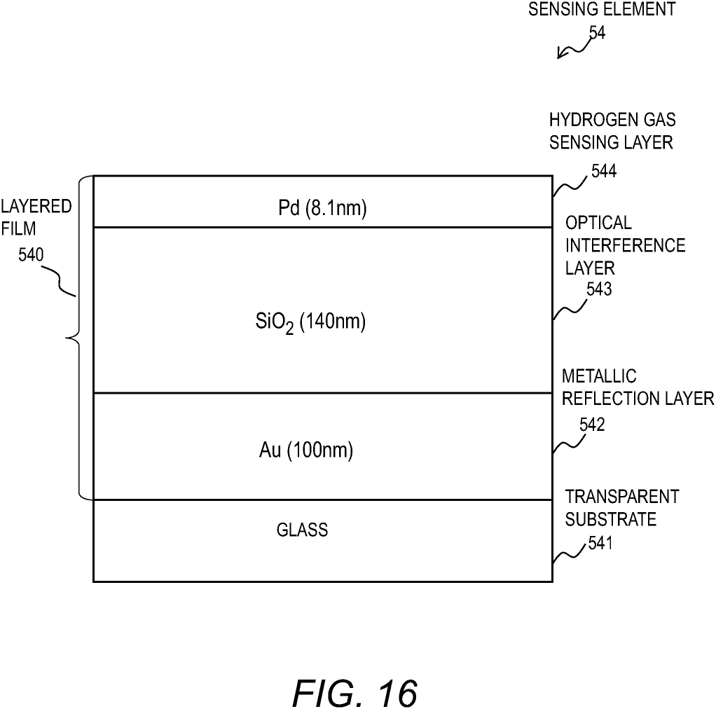

FIG. 16 is a cross-sectional diagram schematically illustrating a configuration example of a sensing element 54 in an embodiment of this specification. The sensing element 54 includes a layered film 540 for sensing hydrogen gas on a transparent glass substrate 541. Instead of the transparent substrate 541, an opaque substrate can be employed. The layered film 540 consists of a metallic reflection layer 542, an optical interference layer 543, and a hydrogen gas sensing layer 544. The metallic reflection layer 542, the optical interference layer 543, and the hydrogen gas sensing layer 544 are stacked on the transparent substrate 541 in this order. This embodiment does not need a prism or an antireflection film on the reverse side of the transparent substrate 541 on which the layered film 540 is not provided.

The metallic reflection layer 542 is an Au thin film of 100 nm in thickness. The optical interference layer 543 is a SiO₂ thin film of 140 nm in thickness. The hydrogen gas sensing layer 544 is a Pd thin film of 8.1 nm in thickness. The hydrogen gas sensing layer 544 also functions as a half mirror layer.

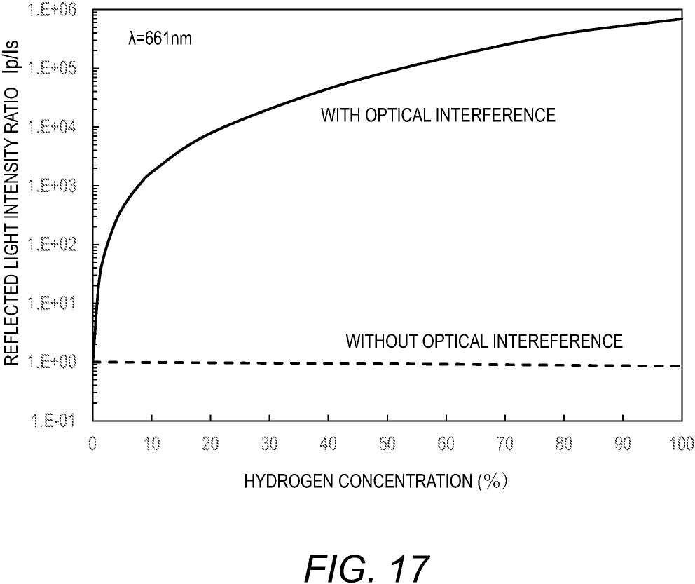

FIG. 17 provides simulation results on an optical characteristic of the layered film 540 illustrated in FIG. 16. The horizontal axis represents hydrogen concentration. The vertical axis is a logarithmic axis and represents the ratio (lp/ls) of the intensity lp of p-polarized light reflected off the layered film 540 to the intensity ls of s-polarized light reflected off the layered film 540. The vertical axis is normalized so that the intensity ratio (lp/ls) takes a value 1 when the hydrogen concentration is 0%. The dashed line represents the variation of the intensity ratio (lp/ls) with respect to the hydrogen concentration in the case of a single Pd layer (100 nm) not having a layered structure. The solid line represents the variation of the intensity ratio (lp/ls) with respect to the hydrogen concentration in the case of the layered film 540 illustrated in FIG. 16. The wavelength λ of the incident light was 661 nm. The calculation result in FIG. 17 indicates that the layered film 540 having optical interference effect sensitively responds to variation of hydrogen concentration.

Although the foregoing embodiments employ a glass substrate as a transparent substrate, the material for the transparent substrate is not limited to this. For Embodiments 1, 2, and 3, a substrate of other material can be employed as far as it is transparent for the light used in measurement. For example, in the case of using a light source of an infrared region, a substate of a semiconductor such as Si or GaAs can be employed.

To illuminate the sensing element from a light source and to guide reflected light off the sensing element to a photodetector, an optical fiber can be used. In this case, a configuration such that a sensing element is integrated with the tip of the optical fiber is available.

The optical signal to be detected by the photodetector device can be modulated by cyclically changing the intensity of light from the light source for illuminating the sensing element to apply synchronized detection or Fourier analysis. Then, the sensitivity in detection improves more because the noise in the optical signal is reduced.

Although the foregoing embodiments have described optical hydrogen gas sensors as examples of optical chemical sensors, the optical chemical sensor of this disclosure is not limited to optical hydrogen gas sensors but is applicable to optical ion sensors for detecting pH, optical gas sensors for detecting a gas, and optical biosensor for detecting DNA or an enzyme. In the cases of application to optical ion sensors for detecting pH, optical gas sensors for detecting a gas, and optical biosensors for detecting DNA or an enzyme, the same effects as described in the foregoing embodiments can be attained.

As set forth above, embodiments of this disclosure have been described; however, this disclosure is not limited to the foregoing embodiments. Those skilled in the art can easily modify, add, or convert each element in the foregoing embodiments within the scope of this disclosure. A part of the configuration of one embodiment can be replaced with a configuration of another embodiment or a configuration of an embodiment can be incorporated into a configuration of another embodiment.

What is claimed is:

1. A sensor system comprising:
a sensing element;
an illumination optical system including a light source, the illumination optical system being configured to obliquely illuminate the sensing element; and
a detector device configured to detect light reflected off the sensing element,
wherein the sensing element includes:
a chemical sensing layer configured to change in an optical characteristic in response to contact with a target substance;
a reflection layer configured to reflect at least part of incident light; and
an intermediate layer located between the reflection layer and the chemical sensing layer, and
wherein the detector device is configured to separately detect p-polarized light and s-polarized light reflected off the sensing element.

2. The sensor system according to claim 1, wherein p-polarized light and s-polarized light reflected off the reflection layer interfere with light reflected off the chemical sensing layer to cause a difference in reflectance of the sensing element for p-polarized light and s-polarized light.

3. The sensor system according to claim 1, wherein the sensing element is composed of non-magnetic materials.

4. The sensor system according to claim 1, wherein the chemical sensing layer is made of a material containing palladium.

5. The sensor system according to claim 1,
wherein the illumination optical system is configured to illuminate the sensing element on the side of the reflection layer;
wherein the reflection layer is a half mirror layer, and
wherein the chemical sensing layer is a total reflection layer.

6. The sensor system according to claim 1,
wherein the illumination optical system is configured to illuminate the sensing element on the side of the chemical sensing layer,
wherein the chemical sensing layer is a half mirror layer, and
wherein the reflection layer is a total reflection layer.

7. The sensor system according to claim 1, further comprising:
a polarization separator disposed between the light source and the sensing element or between the sensing element and the detector device, the polarization separator being configured to separate p-polarized light and s-polarized light from incident light,
wherein the detector device includes:
a first detector configured to detect p-polarized light reflected off the sensing element; and
a second detector different from the first detector, the second detector being configured to detect s-polarized light reflected off the sensing element.

8. The sensor system according to claim 1, further comprising:
a polarization modulator disposed between the light source and the sensing element or between the sensing element and the detector device,
wherein the polarization modulator is configured to:
output one of p-polarized light and s-polarized light out of light received in a first period; and
output the other one of p-polarized light and s-polarized light out of light received in a second period after the first period, and
wherein the detector device is configured to:
detect the one of the p-polarized light and the s-polarized light reflected off the sensing element in the first period; and
detect the other one of the p-polarized light and the s-polarized light reflected off the sensing element in the second period.

9. The sensor system according to claim 1,
wherein one of an antireflection film and a prism is disposed on a side of the sensing element to be illuminated with light from the light source, and
wherein the illumination system is configured so that light from the light source hits the reflection layer through the one of the antireflection film and the prism.

10. The sensor system according to claim 1,
wherein the reflection layer is a tantalum layer, and
wherein the intermediate layer is a nitride layer.

11. A method of detecting a predetermined target substance with a sensing element,
the sensing element including:
a chemical sensing layer configured to change in an optical characteristic in response to contact with the target substance;
a reflection layer configured to reflect at least part of incident light; and
an intermediate layer located between the reflection layer and the chemical sensing layer,
the method comprising:
illuminating the sensing element obliquely;
separately detecting p-polarized light and s-polarized light reflected off the sensing element; and
generating a result of detecting whether the target substance exists based on a comparison result of intensities of the p-polarized light and the s-polarized light.
